(19) United States
(12) Patent Application Publication
(10) Pub. No.: US 2024/0254439 A1
(43) Pub. Date: Aug. 1, 2024

(54) METHOD FOR PREPARING TASTE BUD ORGANOIDS USING ANTERIOR TONGUE EPITHELIAL CELLS

(71) Applicant: Korea University Research and Business Foundation, Seoul (KR)

(72) Inventors: Yong Taek JEONG, Seoul (KR); Mingyeong LEE, Seoul (KR)

(21) Appl. No.: 18/417,028

(22) Filed: Jan. 19, 2024

(30) Foreign Application Priority Data
Jan. 20, 2023 (KR) 10-2023-0008738

(51) Int. Cl. C12N 5/0793

(52) U.S. Cl. CPC C12N 5/062; C12N 2501/105; C12N 2501/115; C12N 2501/155; C12N 2501/165; C12N 2501/41; C12N 2501/415; C12N 2501/727; C12N 2503/00; C12N 2506/097; C12N 2513/00

(57) ABSTRACT

A medium composition for inducing differentiation of taste bud organoids derived from the anterior tongue epithelium, a method for producing taste bud organoids, a taste bud organoid derived from the anterior tongue epithelium, and a method for screening a tastant using the same, wherein the medium composition for inducing differentiation of taste bud organoids derived from the anterior tongue epithelium and the method for producing taste bud organoids using the same may efficiently form the taste bud organoids from tongue epithelial tissue and may control their differentiation into specific types of taste cells. In addition, the taste bud organoids derived from the anterior tongue epithelium formed by the method may maximize the expression of markers for various taste buds and cells that make up taste buds to express salty taste detection proteins, so that they may be used in various fields such as cell sensors and tastant screening.

### TECHNICAL FIELD

The present invention relates to a method for producing taste bud organoids using anterior tongue epithelial cells.

### BACKGROUND ART

Taste buds, the primary taste organs producing gustatory sensory information, are located at the surface of tongue, soft palate, and larygopharyx in the oral cavity. The lingual papillae which located at the posterior regions of tongue, such as circumvallate papilla and foliate papillae, have dozens to hundreds of taste buds, while fungiform papillae at the anterior regions of tongues, have only one or two taste buds. Therefore, circumvallate papillae and foliate papillae generate robust gustatory information while fungiform papillae are responsible for generation of sensory information for various taste qualities as they respond to more diverse taste stimuli.

Taste buds are made up of dozens of cells, which are classified into type 1, type 2, type 3 and type 4 taste cells based on their gene expression, the response to taste stimuli, and cellular morphologies.

Organoids, created by embedding pluripotent and adult stem cells in a three-dimensional extracellular matrix, mimic the specific structure and function of target organs. This is achieved by artificially manipulating the differentiation potential of stem cells. A method for generating taste bud organoids was introduced by culturing taste bud stem cells near the taste buds using conventional organoid culture methods, and these organoids contained each of differentiated type 1, type 2, type 3 and type 4 taste cells.

Most of the currently reported taste bud organoid culture methods use the tissues and cells of circumvallate papillae and foliate papillae, in which taste buds and their stem cells are enriched. As circumvallate papillae and foliate papillae lack the cellular clusters that respond to some taste stimuli (salty taste), so that it is highly likely that taste bud cells derived from these tissues and taste bud organoids comprising the same will not respond to some taste stimuli. In contrast, there has been no reported case of generating taste bud organoids from the tissues and epithelial cells of the anterior tongue, in which fungiform papillae and their taste buds are located.

Against this backdrop, the inventors have successfully developed the present invention by creating a culture method to generate taste bud organoids from anterior lingual epithelial cells, even in cases where taste buds are sparsely distributed. This method could induce differentiation to taste bud and taste cells with efficient yields. The inventors have confirmed that taste bud organoids produced through this method exhibit robust expression of taste bud-specific markers.

### PRIOR ART REFERENCES

### Patent Documents

(Patent Document 1) Korean Patent No. 10-2334245

### DISCLOSURE

### Technical Problem

The present invention aims to solve the above-described problems and other problems associated therewith.

It is an exemplary object of the present invention to provide a medium composition for inducing differentiation of taste bud organoids derived from the anterior tongue epithelium.

It is another exemplary object of the present invention to provide a method for producing taste bud organoids derived from the anterior tongue epithelium.

It is another exemplary object of the present invention to provide a taste bud organoid derived from the anterior tongue epithelium, prepared by the production method.

It is another exemplary object of the present invention to provide a method for screening a tastant, comprising the steps of:
treating the taste bud organoid with a target substance;
determining whether the target substance is a tastant.

The technical problems to be achieved according to the technical idea of the invention disclosed in the present specification are not limited to the above-mentioned problems to be solved, and other problems not mentioned will be clearly understood by those skilled in the art from the description below.

### Technical Solution

These will be described in detail as follows. On the other hand, each description and embodiment disclosed in the present application may also be applied to each other description and embodiment. That is, all combinations of the various elements disclosed in this application fall within the scope of this application. In addition, it cannot be seen that the scope of the present application is limited by the specific descriptions described below.

In one aspect for achieving the above objects, the present invention provides a medium composition for inducing differentiation of taste bud organoids derived from the anterior tongue epithelium.

The medium composition for inducing differentiation of taste bud organoids derived from the anterior tongue epithelium according to the present invention (hereinafter referred to as differentiation culture medium 1) comprises R-spondin 1, noggin, FGF2, IGF1, smoothened agonist (SAG), and Y-27632.

The R-spondin 1 is a secretory protein known as a Wnt agonist and may be included in the medium composition in the form of conditioned medium or recombinant protein, and the concentration of R-spondin 1 in the medium composition may be appropriately selected to control the composition of each type of taste cell.

As an example, conditioned medium of a cell line stably expressing R-spondin 1 may be included at a level of 2 to 50%, and may be included at a level of specifically 2 to 20% or 10 to 30%, more preferably 10 to 20%, based on the total volume of the medium composition. In addition, when R-spondin 1 is added to the medium composition in the form of a recombinant protein, it may be specifically included at a concentration of 10 to 1000 ng/ml and may be more preferably included at, but is not limited to, a concentration of 10 to 500 ng/ml, 25 to 500 ng/ml, 10 to 300 ng/ml or 10 to 100 ng/ml.

In addition, the R-spondin 1 in the medium composition of the present invention may be included in the medium composition by replacing it with R-spondin 2, R-spondin 3, R-spondin 4, or the like, which belong to the R-spondin family.

The noggin is a protein involved in the development of many body tissues, including nervous tissue, muscle and bone, and may be included in the medium composition in the form of conditioned medium or recombinant protein, and the concentration of noggin in the medium composition may be appropriately selected to control the composition of each type of taste cell.

As an example, conditioned medium of a cell line stably expressing noggin may be included at a level of 1 to 20%, and may be included at a level of specifically 2 to 10% or 5 to 20%, more preferably 5 to 15%, based on the total volume of the medium composition. In addition, when noggin is added to the medium composition in the form of a recombinant protein, it may be specifically included at a concentration of 10 to 200 ng/ml and may be more preferably included at, but is not limited to, a concentration of 10 to 150 ng/ml, 50 to 150 ng/ml or 50 to 100 ng/ml.

In addition, the noggin in the medium composition of the present invention is used as an inhibitor of BMP receptors, and may be included in the medium composition by replacing it with dorsomorphin, chordin, gremlin, short gastrulation (sog), follistatin, differential screening-selected gene aberrant in neuroblastoma (DAN), cerberus, Dante, or the like, which are inhibitors of BMP receptors known in the art.

The fibroblast growth factor 2 (FGF2) and insulin-like growth factor 1 (IGF1) are proteins that regulate cell growth, proliferation and differentiation, and the concentration of FGF2 and IGF1 in the medium composition may be appropriately selected to regulate cell growth, proliferation and differentiation.

As an example, the FGF2 and IGF1 may be included at a concentration of 10 to 200 ng/ml and may be more preferably included at, but are not limited to, a concentration of 10 to 100 ng/ml, 20 to 100 ng/ml or 50 to 100 ng/ml.

In addition, the FGF2 in the medium composition of the present invention may be included in the medium composition by replacing it with FGF1, FGF3, FGF4, FGF5, FGF6, FGF7, FGF8, FGF9, FGF10, or the like, or an analogue of FGF2, and the IGF1 may also be included in the medium composition by replacing it with IGF2 or an analogue of IGF1.

The smoothened agonist (SAG) is an active substance in the sonic hedgehog signaling pathway, and the concentration of SAG in the medium composition may be appropriately selected to regulate the relevant signaling pathway.

As an example, the SAG may be included at a concentration of 0.1 to 20 μM and may be more preferably included at, but is not limited to, a concentration of 0.5 to 10 μM, 1 to 10 μM or 0.5 to 5 μM.

In addition, the SAG in the medium composition of the present invention is added to stimulate the hedgehog signaling pathway, and may be included in the medium composition by replacing it with SAG HCl, purmorphamine, or the like, which are analogues of SAG known to have the same mechanism of action in the art.

Y-27632 (dihydrochloride) is a compound that inhibits the Rho/Rock signaling pathway and has the structure of following Formula (1), and the concentration of Y-27632 in the medium composition may be appropriately selected to efficiently proliferate and differentiate cells that make up taste buds.

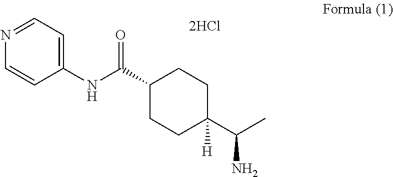 [SMILES: C[C@@H](N)[C@H]1CC[C@H](C(=O)Nc2ccncc2)CC1]

As an example, the Y-27632 may be included at a concentration of 0.1 to 20 μM and may be more preferably included at, but is not limited to, a concentration of 0.5 to 20 μM, 1 to 10 μM or 5 to 10 μM.

Another medium composition for inducing differentiation of taste bud organoids derived from the anterior tongue epithelium according to the present invention (hereinafter referred to as differentiation culture medium 2) comprises R-spondin 1, noggin, FGF2, IGF1, smoothened agonist (SAG), and CHIR99021.

CHIR99021 (laduviglusib) is a compound that inhibits GSK-3a/B and activates the Wnt/B-catenin signaling pathway and has the structure of following Formula (2), and the concentration of CHIR99021 in the medium composition may be appropriately selected to efficiently activate the Wnt/B-catenin signaling pathway.

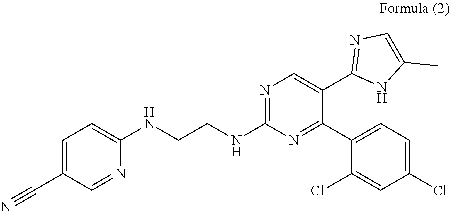 [SMILES: Cc1cnc(-c2cnc(NCCNc3ccc(C#N)cn3)nc2-c2ccc(Cl)cc2Cl)[nH]1]

As an example, the CHIR99021 may be included at a concentration of 0.1 to 20 μM and may be more preferably included at, but is not limited to, a concentration of 0.5 to 20 μM, 1 to 10 μM or 1 to 5 μM.

In addition, the CHIR99021 in the medium composition of the present invention is added to stimulate the Wnt/B-catenin signaling pathway, and may be included in the medium composition by replacing it with Wnt proteins (Wnt1, Wnt2, Wnt3, Wnt4, Wnt5, Wnt6, Wnt7, Wnt8, Wnt9, Wnt10, Wnt11, Wnt12), LiCl, or the like, which are known to have the same mechanism of action in the art. When WNT protein is included instead of the CHIR99021, conditioned medium of a cell line stably expressing Wnt protein may be included at a level of 2 to 50%, and may be included at a level of specifically 2 to 20% or 10 to 50%, more preferably 10 to 30%, based on the total volume of the medium composition. In addition, when LiCl is added to the medium composition, it may be specifically included at a concentration of 1 to 10 mM and may be more preferably included at, but is not limited to, a concentration of 1 to 5 mM.

The differentiation culture medium 2 may further comprise SU5402.

The SU5402 is a multi-target receptor tyrosine kinase inhibitor compound targeting VEGFR2, FGFR1, PDGFRβ, and the like, and has the structure of following Formula (3), and the concentration of SU5402 in the medium composition may be appropriately selected to efficiently inhibit the FGF receptor signaling pathway.

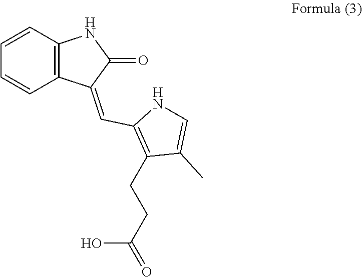 [SMILES: Cc1c[nH]c(/C=C2\C(=O)Nc3ccccc32)c1CCC(=O)O]

As an example, the SU5402 may be included at a concentration of 0.1 to 20 μM and may be more preferably included at, but is not limited to, a concentration of 0.5 to 20 μM, 1 to 10 μM or 5 to 10 μM.

In addition, the SU5402 in the medium composition of the present invention is added to inhibit the FGF receptor signaling pathway, and may be included in the medium composition by replacing it with cediranib, FIIN1 hydrochloride, nintedanib, PD161570, PD166285 dihydrochloride, PD173074, SU6668, or the like, which are selective inhibitors of FGF receptor known in the art.

The term “taste bud” as used herein refers to cells distributed in the oral cavity, such as the tongue or soft palate, and is known to be able to perform the role of sensing taste in contact with food, and specifically to sense sweet, bitter, salty, sour and umami tastes.

The term “organoid” as used herein refers to an ex vivo 3D cellular cluster composed of primary tissues, tissue subunits or single cells (e.g., stem cells). Organoids have the characteristics capable of self-renewal and self-organization, and may implement phenotypes and functions similar to native tissues, so that they may also be called “small organ-like organs” or “organ analogues.”

In another aspect for achieving the above objects, the present invention comprises a method for producing taste bud organoids derived from the anterior tongue epithelium, comprising the steps of: (a) extracting cells from the epithelial lamina of mouse tongue tissue; (b) culturing the extracted cells in a first medium composition; and (c) replacing the first medium composition with a second medium composition and then culturing the cultured cells in the second medium composition.

The first medium composition comprises R-spondin 1, noggin, FGF2, IGF1, smoothened agonist (SAG) and Y-27632, and the second medium composition comprises R-spondin 1, noggin, FGF2, IGF1, smoothened agonist (SAG), CHIR99021 and SU5402.

“R-spondin 1,” “noggin,” “FGF2,” “IGF1,” “SAG,” “Y-27632,” “CHIR99021,” “SU5402,” “taste bud” and “organoids” are as described above.

The term “culturing” as used herein refers to the ex vivo proliferation, growth, maintenance and differentiation of cells isolated from living organisms, their two-dimensional or three-dimensional clusters, tissues, or parts of tissues. Accordingly, the term “culturing” is meant to encompass the entire process of obtaining the desired material in an artificial environment using starting materials (cells, tissues or tissue analogues), and “composition for culturing” is a concept including “composition for proliferating,” “composition for growing,” “composition for maintaining,” and “composition for inducing differentiation.”

The process of treating mouse tongue tissue with a dispase enzyme to separate the epithelial lamina in step (a) may be performed by treating the mouse tongue tissue with dispase II enzyme or injecting it into the tissue in order to effectively isolate the tongue epithelial cells therefrom. As an example, according to Example 1.1 of the present invention, dispase II enzyme (2 mg/mL) was injected into the space between the mouse tongue epithelium and subepithelial connective tissue and reacted at 37° ° C. for 30 minutes or more to peel the epithelial layer off from the subepithelial connective tissue, thereby separating the epithelial lamina therefrom.

The process of extracting cells in step (a) is a process of separating single cells that make up the separated epithelial lamina therefrom, and as an example, according to Example 1.1 of the present invention, the separated epithelial lamina was cut into as small an area as possible using tools such as scissors or knives, and then reacted in a trypsin/EDTA solution at 37° C. for 30 minutes or more, and subjected to pressure by pipetting to extract cells from the tissue.

The extracted cells in step (b) may be cultured by an organoid culture method known in the art, and as an example, according to Example 1.1 of the present invention, after centrifugation of the extracted cells or a single cell suspension containing the extracted cells, the cell pellets were embedded in an extracellular matrix such as Matrigel and cultured.

The method for producing taste bud organoids according to the present invention may further comprise the step of confirming the expression of markers for taste bud cells in cells isolated from anterior tongue epithelial tissue.

The markers for taste bud cells are markers that may confirm whether cells derived from tongue epithelial tissue may differentiate into taste bud cells to perform the function of taste buds, and include, but are not limited to, for example, TRPM5, Car4, NTPdase, PLCB2, GNAT3, NCAM, SNAP25, and the like.

The culture period in step (b) or step (c) may be appropriately selected to control the composition of each type of taste cell, and specifically, the culture in step (b) may be performed in the first medium composition for 2 days or more, specifically 2 to 10 days, more preferably 2 to 5 days, and the culture in step (c) may be performed in the second medium composition for 5 days or more, specifically 5 to 15 days, more preferably 7 to 15 days, but is not limited thereto.

In another aspect for achieving the above objects, there is provided a taste bud organoid derived from the anterior tongue epithelium, prepared by the production method of the present invention.

In another aspect for achieving the above objects, the present invention provides a method for screening a tastant, comprising the steps of: treating the taste bud organoid with a target substance; and determining whether the target substance is a tastant.

The taste bud organoids of the present invention are formed from the anterior tongue epithelium (including fungiform papillae), which includes cellular clusters that respond to salty taste, and are not formed from the posterior tongue (including circumvallate papillae and foliate papillae), which lacks the cellular clusters, and thus, they include taste bud cells in which proteins that may detect salty taste were expressed. Therefore, when the taste bud organoids of the present invention are used as a cell sensor, screening for various tastants, including salty taste, is possible.

In the present invention, it is possible to determine whether the target substance is a tastant by measuring the change in the expression level of the taste sensing proteins of the taste bud organoid treated with the target substance.

In one embodiment of the present invention, it is possible to determine whether the target substance is a tastant by measuring the response of the taste bud organoids to the target substance by changes in cell activity using electrophysiological techniques (e.g., patch clamp), calcium imaging, and the like.

### Advantageous Effects

The medium composition for inducing differentiation of taste bud organoids derived from the anterior tongue epithelium according to the present invention and the method for producing taste bud organoids using the same may induce differentiation of tongue epithelial cells, which are sparse in taste buds and taste bud stem cells, into taste buds and cells that make up taste buds.

In addition, the method for producing taste bud organoids according to the present invention may form taste bud organoids at an efficient yield from a relatively small number of tissues compared to taste bud organoids obtained from a limited number of circumvallate papillae and foliate papillae, and may control their differentiation into specific types of taste cells.

In addition, the taste bud organoids derived from the anterior tongue epithelium formed by the method of the present invention may maximize the expression of markers for various taste buds and cells that make up taste buds to express salty taste sensing proteins, so that they may be used in various fields such as cell sensors and tastant detection.

However, the effects according to an embodiment of the technology disclosed in the present specification are not limited to those mentioned above, and other effects not mentioned will be clearly understood by those skilled in the art from the description below.

### DESCRIPTION OF DRAWINGS

In order to more fully understand the drawings cited in present specification, a brief description of each drawing is provided.

FIG. 1 shows the process of producing organoids derived from the anterior tongue.

FIG. 2 shows the results of confirming the culture efficiency of organoids when adding SAG to various culture conditions in culturing taste bud organoids from the anterior tongue epithelial tissue.

FIG. 3 shows the results of confirming the change in expression efficiency of markers for cells that make up taste buds in the organoids and the differentiation efficiency into cells that make up taste buds when adding SAG to various culture conditions in culturing taste bud organoids from the anterior tongue epithelial tissue.

FIG. 4 shows the results of confirming the change in expression efficiency of markers for cells that make up taste buds in the organoids and the differentiation efficiency into cells that make up taste buds through pharmacological regulation of Wnt and Notch signals in SAG-containing basic culture medium, when culturing taste bud organoids derived from the anterior tongue epithelium.

FIG. 5 shows the results confirming the optimization of the expression efficiency of markers for cells that make up taste buds in the organoids and the differentiation efficiency into cells that make up taste buds for each period of treatment with CHIR (cultured in culture medium containing SAG and CHIR), when culturing taste bud organoids derived from the anterior tongue epithelium.

FIG. 6 shows the results of confirming the optimization of the expression efficiency of markers for cells that make up taste buds in the organoids and the differentiation efficiency into cells that make up taste buds through the addition of CHIR to the SAG-containing basic culture medium after the first 3 days of culture and manipulation of growth differentiation factor signaling thereafter, when culturing taste bud organoids derived from the anterior tongue epithelium.

### BEST MODE

Hereinafter, the present invention will be described in more detail through the following examples. However, these examples are only for illustrating the present invention, and the scope of the present invention is not limited to these examples only.

### Example 1: Production of Anterior Tongue Organoids

a. Obtaining Anterior Tongue Epithelial Cells

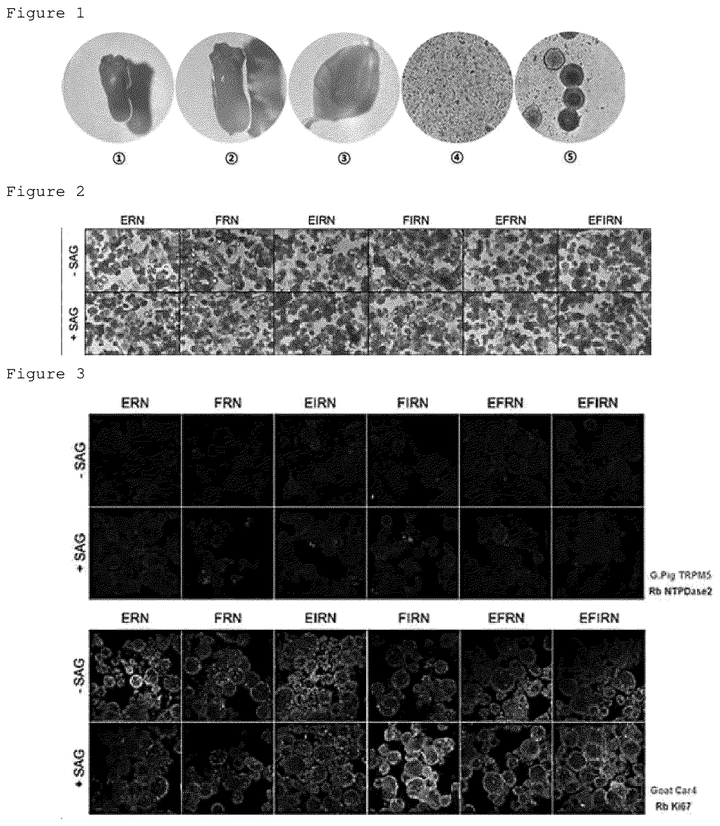

The tongue was collected from a mouse, and dispase II enzyme (2 mg/mL) (Roche, cat #4942078001) diluted in physiological saline was injected into the space between the epithelium and subepithelial connective tissue to expand the tongue tissue ((2) in FIG. 1), and the expanded tongue tissue was cultured in dispase II enzyme dilution at 37° C. for 30 minutes. The epithelial layer is peeled off from the subepithelial connective tissue to separate the epithelial lamina therefrom, and it is cut into as small an area as possible using tools such as sterilized surgical scissors or a knife in trypsin/EDTA (Gibco, cat #25200072) solution. The finely cut tissues were cultured at 37° C. for 30 minutes. Thereafter, the finely cut tissues are subjected to pressure by continuous pipetting to extract cells from the tissue and form a single cell suspension. The cell pellets obtained by centrifuging this suspension are embedded in an extracellular matrix such as Matrigel at an appropriate ratio.

b. Production of Organoids Derived from Anterior Tongue Epithelial Cells

All culture media are based on basic culture medium (DMEM/F12+1×GlutaMax+1×HEPES+1×N2 supplement+1×B27 supplement+1×penicillin/streptomycin).

The single cells embedded in the Matrigel were cultured in differentiation medium 1 (FIRNY+SAG) for 4 days, then cultured for 10 days, while replacing the culture medium with differentiation medium 2 (FIRN+SAG+CHIR_SU5402) and changing the medium culture every 2-3 days.

TABLE 1

| | |
|---|---|
| Basic culture | DMEM/F12 (Gibco, cat #11320033) |
| medium | 1x GlutaMax (Gibco, cat #35050-061) |
| | 1x HEPES (Sigma, cat #83264) |
| | 1x N2 supplement (Gibco, cat #17502-048) |
| | 1x B27 supplement (Gibco, cat #17504-044) |
| | 1x P/S (Gibco, cat # 15140-122) |
| Differentiation | 20% R-spondin 1 |
| culture | 10% Noggin |
| medium 1 | 50 ng/mL FGF2 |
| (FIRNY* + SAG) | 50 ng/mL IGF1 (BioLegend, cat #590908) |
| | 1 μM SAG (Abcam, cat #ab142160) |
| | 10 μM Y-27632 (Cayman chemical, cat # |
| | Cay10005583-50) |
| Differentiation | 20% R-spondin 1 |
| culture | 10% Noggin |
| medium 2 | 50 ng/mL FGF2 |
| (FIRN + SAG + | 50 ng/mL IGF1 |
| CHIR + SU5402) | 1 μM SAG |
| | 3 μM CHIR99021 (Sigma, cat #SML1046-5MG) |
| | 10 μM SU5402 (Sigma, cat #SML0443-5MG) |

*F; FGF2, I; IGF1, R; R-spondin 1, N; noggin, CHIR; CHIR99021

### Example 2: Optimization of Culture Efficiency of Organoids by Culture Conditions

### 2.1. Confirmation of Culture Efficiency of Organoids by Addition of SAG

In culturing taste bud organoids from anterior tongue epithelial tissue, there was no change in the growth efficiency or size of the organoid itself (FIG. 2) when EGF, FGF2 and IGF1 were added alone or in combination to the basic culture medium supplemented with R-spondin1 and noggin and when cultured by adding SAG under all of the above conditions, but differentiation into type 1, type 2 and type 3 taste cells was confirmed when SAG was added. In particular, it was confirmed by immunofluorescence staining that differentiation efficiency increased when FGF2 was added alone or in combination with EGF and IGF1, and differentiation efficiency was maximized when FGF2 and IGF1 were added in combination (FIG. 3).

### 2.2. Confirmation of Differentiation Efficiency by Pharmacological Regulation of Wnt and Notch Signals

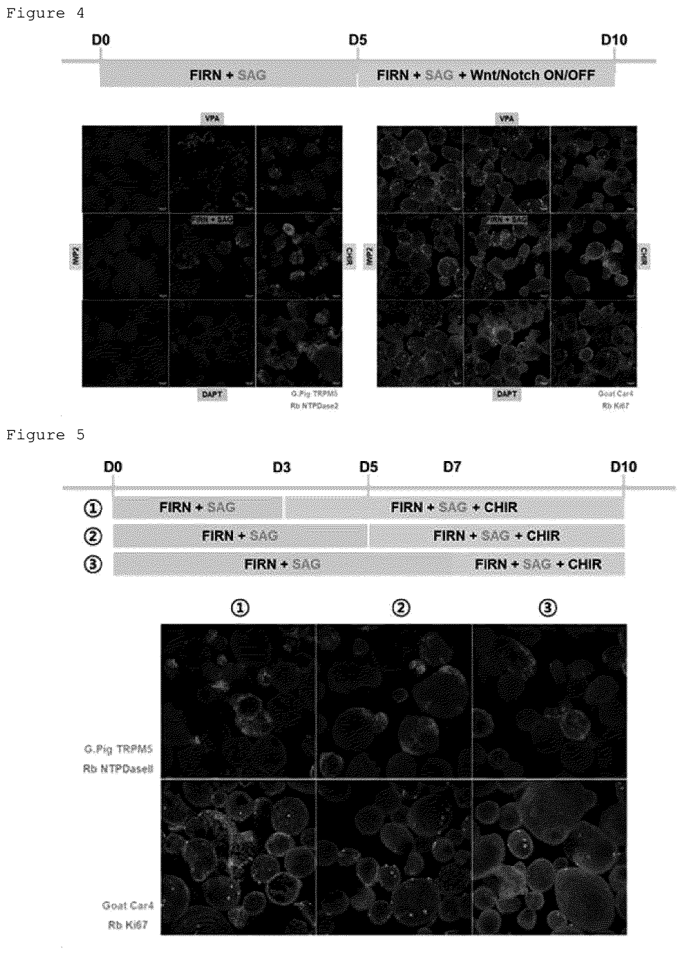

In culturing taste bud organoids from anterior tongue epithelial tissue, it was confirmed by immunofluorescence staining that when SAG-containing basic culture medium was treated with CHIR99021 (CHIR), which stimulates the Wnt and Notch signaling pathways, the differentiation efficiency into type 1, type 2 and type 3 taste cells all increased (FIG. 4). In addition, as a result of confirming the differentiation efficiency into taste cells by CHIR-treated time when treated with CHIR on days 3, 5 or 7, it was confirmed by immunofluorescence staining that the earlier treatment with CHIR, the higher the differentiation efficiency into type 1, type 2 and type 3 taste cells (FIG. 5).

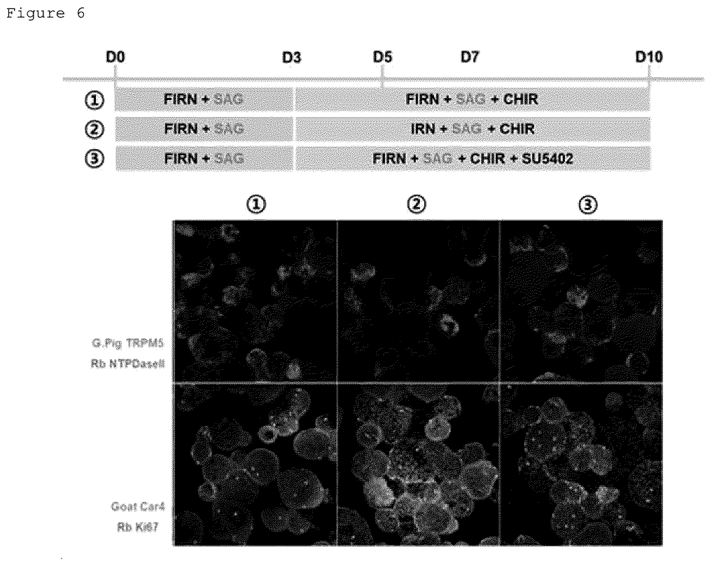

In order to confirm the optimization of the expression efficiency of markers for cells that make up taste buds in organoids and the differentiation efficiency into cells that make up taste buds by components other than CHIR, CHIR was added after the first 3 days of culture, and organoids were cultured in culture medium in which FGF was removed or SU5402 was additionally added. As a result, it was confirmed by immunofluorescence staining that when FGF was removed or SU5402 was additionally input after 3 days of culture, the differentiation efficiency into type 2 and type 3 taste cells increased (FIG. 6).

From the above description, those of ordinary skill in the technical art to which the present invention pertains will understand that the present invention may be implemented in other specific forms without changing the technical spirit or essential characteristics thereof. In this regard, it should be understood that the examples described above are illustrative and not restrictive in all respects. It should be construed that all changes or modifications derived from the meaning and scope of the claims to be described later rather than the detailed description and their equivalent concepts are included in the scope of the present invention.

What is claimed is:

1. A medium composition for inducing differentiation of taste bud organoids derived from the anterior tongue epithelium, comprising R-spondin 1, noggin, FGF2, IGF1, smoothened agonist (SAG), and Y-27632.

2. The medium composition according to claim 1, wherein Y-27632 is removed and CHIR99021 is further comprised.

3. The medium composition according to claim 2, further comprising SU5402.

4. The medium composition according to claim 2, wherein the CHIR99021 is comprised at a concentration of 0.1 to 10 M.

5. The medium composition according to claim 3, wherein the SU5402 is comprised at a concentration of 0.1 to 20 μM.

6. A method for producing taste bud organoids derived from the anterior tongue epithelium, comprising the steps of:
extracting cells from the epithelial lamina of mouse tongue tissue;
culturing the extracted cells in a first medium composition, wherein the first medium composition comprises R-spondin 1, noggin, FGF2, IGF1, smoothened agonist (SAG), and Y-27632; and
replacing the first medium composition with a second medium composition and then culturing the cultured cells in the second medium composition, wherein the second medium composition comprises R-spondin 1, noggin, FGF2, IGF1, smoothened agonist (SAG), CHIR99021, and SU5402.

7. The method according to claim 6, wherein the step of extracting cells in step (a) comprises treating mouse tongue tissue with a dispase enzyme to separate the epithelial lamina and extracting cells therefrom.

8. The method according to claim 6, wherein the extracted cells in step (b) are embedded in an extracellular matrix and cultured.

9. The method according to claim 6, further comprising the step of confirming the expression of markers for taste bud cells in the cultured cells.

10. The method according to claim 6, wherein the culturing in step (b) is performed in the first medium composition for 2 to 5 days.

11. The method according to claim 6, wherein the culturing in step (c) is performed in the second medium composition for 7 to 15 days.

12. A taste bud organoid derived from the anterior tongue epithelium, produced by the method according to claim 6.

13. A method for screening a tastant, comprising the steps of:
treating the taste bud organoid according to claim 12 with a target substance; and
determining whether the target substance is a tastant.
